(19) United States
(12) Patent Application Publication
(10) Pub. No.: US 2013/0302808 A1
(43) Pub. Date: Nov. 14, 2013

(54) PREDICTING CANCER OUTCOME

(71) Applicants: George Vasmatzis, Oronoco, MN (US); John C. Cheville, Pine Island, MN (US); R. Jeffrey Karnes, Rochester, MN (US); Cristiane M. Ida, Rochester, MN (US); Jan Marie A. Willard, Weslaco, TX (US)

(72) Inventors: George Vasmatzis, Oronoco, MN (US); John C. Cheville, Pine Island, MN (US); R. Jeffrey Karnes, Rochester, MN (US); Cristiane M. Ida, Rochester, MN (US); Jan Marie A. Willard, Weslaco, TX (US)

(73) Assignee: MAYO FOUNDATION FOR MEDICAL EDUCATION AND RESEARCH, Rochester, MN (US)

(21) Appl. No.: 13/811,555

(22) PCT Filed: Jul. 12, 2011
(86) PCT No.: PCT/US11/43703

§ 371 Date: Jul. 18, 2013

Related U.S. Application Data
(60) Provisional application No. 61/366,608, filed on Jul. 22, 2010.

(51) Int. Cl. C12Q 1/68; G01N 33/68

(52) U.S. Cl. CPC C12Q 1/6886; G01N 33/6875
USPC 435/6.12

(57) ABSTRACT

This document provides methods and materials related to assessing prostate cancer in mammals. For example, methods and materials for using ERG expression levels and TOP2A expression levels to identify mammals (e.g., male human patients) as being susceptible to a poor prostate cancer outcome are provided.

### CROSS-REFERENCE TO RELATED APPLICATIONS

This application claims the benefit of priority to U.S. Provisional Application Ser. No. 61/366,608, filed on Jul. 22, 2010.

### BACKGROUND

1. Technical Field

This document relates to methods and materials involved in assessing prostate cancer in male mammals. For example, this document provides methods and materials for determining whether a mammal having prostate cancer is susceptible to a poor outcome.

2. Background Information

Two of the major decision-making points related to therapy for patients with prostate cancer are at the time of biopsy and after radical prostatectomy. Positive biopsies contain a small portion of the tumor for diagnosis of aggressiveness. Based on morphological parameters such as Gleason score, patients are given three choices. In the case of benign or less aggressive disease, the choices are either watchful waiting or surgery. Immediate surgery is recommended in intermediate situations to prevent further advance. Radiation or hormonal therapies are recommended if the cancer has metastasized and surgery would not help. After surgery, when the bulk of the tumor is available for more thorough diagnosis, decisions still have to be made concerning additional therapies such as radiation or hormonal treatment that could depend on the aggressiveness of the disease. Determining disease aggressiveness can be important for clinical decisions towards the management of prostate cancer patients.

### SUMMARY

This document provides methods and materials related to assessing prostate cancer in mammals (e.g., human males). For example, this document provides methods and materials for identifying a mammal as having an aggressive form of prostate cancer. Gleason grade, stage, ploidy, and margin status are major descriptors of prostate cancer aggressiveness and are thought to be important in determining cancer management. These clinicopathological parameters can have significant drawbacks. For example, Gleason grading is heavily dependent upon the visual interpretation (Gleason, Hum. Pathol., 23:273-279 (1992); and Gleason and Vacurg (1977) Histologic grading and clinical staging of prostatic carcinoma. In Urologic pathology: the prostate (Tannenbaum M., ed.) Lea & Febiger, Philadelphia, Pa. 171-213). In addition, the level of resolution of this grading system appears to be too coarse as it is dependent on architectural pattern rather than cytologic changes. This concern is supported by the high level of uncertainty regarding the clinical outcome of patients with intermediate Gleason grades.

As described herein, prostate cancer patients who lack ERG mRNA expression (ERG(−)) and contain an elevated level of TOP2A expression or elevated MIB1 can be classified as being susceptible to a poor prostate cancer outcome. Identifying cancer patients who have a poor prognosis can allow such patients, who are at risk for progression and prostate cancer death, to be offered more aggressive therapy earlier.

In general, one aspect of this document features a method for assessing prostate cancer. The method comprises, or consists essentially of, (a) detecting reduced or no ERG expression in a mammal having prostate cancer, (b) detecting the presence of an elevated level of expression of TOP2A nucleic acid, or a polypeptide encoded by the nucleic acid, in the mammal, and (c) classifying the mammal as being susceptible to a poor prostate cancer outcome based at least in part on the reduced or no ERG expression and the presence of the elevated level. The mammal can be a human. The reduced or no ERG expression can be detected based on a lack of ERG expression. The elevated level can be detected in prostate tissue. The elevated level can be detected using PCR or in situ hybridization. The elevated level can be detected using immunohistochemistry. The poor prostate cancer outcome can comprise systemic progression within five years of prostatectomy. The poor prostate cancer outcome can comprise prostate cancer death. The mammal can comprise a human treated by radical prostatectomy.

In another aspect, this document features a method for assessing prostate cancer. The method comprises, or consists essentially of, (a) detecting the presence of an elevated level of expression of TOP2A nucleic acid, or a polypeptide encoded by the nucleic acid, by prostate cancer cells of a mammal, wherein the cells have reduced or no ERG expression, and (b) classifying the mammal as being susceptible to a poor prostate cancer outcome. The method can comprise detecting reduced or no ERG expression by the cells. The reduced or no ERG expression can be detected based on a lack of ERG expression. The mammal can be classified as being susceptible to the poor prostate cancer outcome based at least in part on the reduced or no ERG expression and the presence of the elevated level. The mammal can be a human. The elevated level can be detected using PCR or in situ hybridization. The elevated level can be detected using immunohistochemistry. The poor prostate cancer outcome can comprise systemic progression within five years of prostatectomy. The poor prostate cancer outcome can comprise prostate cancer death. The mammal can comprise a human treated by radical prostatectomy.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1: (A, B) TOP2A immunostaining quantitation. (A) The tumor was manually circled on the slide. 6.3×. (B) Using the digital imaging analysis software, the tumor was outlined by a thin black line. 50×. (C, D) TOP2A LI as a predictor of SP and death of prostate cancer: (C) ERG(−) Gleason 4+4 adenocarcinoma from a patient that died from metastatic prostate cancer (case) showing TOP2A LI of 4.5% compared to a (D) ERG(−) Gleason 5+5 adenocarcinoma from patient that was alive and free of disease 7 years after RP (control) showing TOP2A LI of 0.4%. 100×. (E, F) MIB-1 vs. TOP2A LIs in an ERG(−) Gleason 4+4 adenocarcinoma from a patient that was alive and free of disease (control): (E) high MIB-1 LI of 6.24%. 50×. vs. (F) low TOP2A LI of 0.15%, 50×.

FIG. 2: (A) ROC analysis for TOP2A LI. The AUC was 0.75 for all patients (middle black line), 0.81 for 140 patients with ERG negative tumors (grey line) and 0.67 for 111 ERG positive cases (lower black line). (B). Scatter gram of TOP2A LI for cases and controls relative to ERG status.

FIG. 3: (A) ROC analysis for MIB1 LI. The AUC was 0.78 for 140 patients with ERG negative tumors (upper black line) and 0.68 for 111 ERG positive cases (lower black line). (B). Scatter gram of MIB1 LI for cases and controls relative to ERG status.

FIG. 4: Time to systemic progression (SP) related to ERG status and TOP2A Labeling Index. All patients in this study (cases and controls) were used to produce these plots. The percent of patients that developed SP was plotted against “Time to SP” for the three conditions labeled and lower-risk (TOP2A LI<1), intermediate (1<TOP2A LI<2,) and high-risk (TOP2A LI>2) groups. Plot (A) is restricted to patients with ERG(−) tumors, and plot (B) is restricted to patients with ERG(+)tumors.

FIG. 5: TOP2A and ERG status related to response to adjuvant therapy. Percent of patients that developed SP was plotted against “Time to SP” according to ERG status for all cases (A and B) and for those with TOP2A LI>2 (C and D). Curves of the treated group are grey, while curves of the non-treated group are black. The number of patients corresponding to each curve are noted next to the curve. (A) ERG(+) patients that developed SP, (B) ERG(−) patients that developed SP, (C) ERG(+) patients that developed SP and had a TOP2A LI >2, and (D) ERG(−) patients that developed SP and had a TOP2A LI>2.

### DETAILED DESCRIPTION

This document provides methods and materials related to assessing prostate cancer in mammals. For example, this document provides methods and materials for identifying prostate cancer patients having a high level of susceptibility to a poor prostate cancer outcome by determining whether or not a biological sample (e.g., prostate tissue sample) from the mammal (e.g., a male human) contains cells lacking an ERG mRNA expression and contains cells having an elevated level TOP2A expression. As described herein, if a mammal with prostate cancer contains prostate cancer cells lacking an ERG mRNA expression and having an elevated level of TOP2A, then the mammal can be classified as being susceptible to a poor outcome or as having aggressive prostate cancer.

The term “aggressive” as used herein refers to the invasive and metastatic activity of a cancer. For example, an aggressive prostate cancer is more invasive and metastatic than a less aggressive prostate cancer. Aggressive cancers can produce adverse changes in a mammal's overall health to a greater extent than if that cancer were not aggressive. A mammal with an aggressive prostate cancer can, for example, experience bladder obstruction problems to a greater extent than if that prostate cancer were not aggressive. Other adverse changes in overall health include, without limitation, edema, mineral and vitamin deficiencies, increased risk of infection, loss of appetite, depression, enlargement of organs such as lymph nodes, and pain associated with metastasis. Aggressive cancers can increase mortality to a greater extent than less aggressive cancers. For example, aggressive prostate cancer can cause a poor outcome such as systemic progression within five years of prostatectomy. To assess the aggressiveness of prostate cancer in a mammal or to identify a mammal having prostate cancer as being susceptible to a poor prostate cancer outcome, the ERG mRNA expression status and TOP2A expression level can be analyzed in a sample from the mammal. Examples of an ERG nucleic acid include, without limitation, those having the sequence set forth in GenBank® Accession No. NM_{—}004449.4, NM_{—}001136154.1, NM_{—}001136155.1, or NM_{—}182918.3 (GI Nos. 209954801, 209954796, 209954807, and 209954798, respectively). Human TOP2A nucleic acid can have the sequence set forth in GenBank® Accession No. NM_{—}001067.2 (GI No. 19913405), and a human TOP2A polypeptide can have the sequence set forth in GenBank® Accession No. NP_{—}001058.2 (GI No. 19913406).

The presence or absence of ERG can be determined using any appropriate technique. For example, nucleic acid sequencing techniques, PCR-based techniques, and nucleic acid-based mutation detection techniques can be performed to detect the presence or absence of ERG. The presence of a functional (able to generate mRNA) ERG can be determined by assessing overexpression of ERG mRNA levels. For example, methods described elsewhere can be used to indicate the presence of ERG based on ERG mRNA overexpression (Kosari et al., Clin. Cancer Res., 14:1734-1743 (2008)). For example, quantitative PCR can be performed for ERG mRNA expression. Briefly, H&E sections of a prostate tissue sample can be reviewed, and the area of tumor can be identified (e.g., circled). One or more tissue sections (e.g., ten-micron sections) can be obtained, prepared under RNase free conditions, and deparaffinized in xylene. The tumor can be scraped from the slide based on the circled template from the H&E slide and can be placed in a tube (e.g., a 1.5-mL tube) containing a digestion buffer (e.g., a digestion buffer from the RecoverAll kit, Ambion, Austin, Tex.). Total RNA can be isolated and treated with DNase. The amount of RNA can be measured. Reverse transcription (RT) can be performed, and quantitative PCR can be performed using, for example, an ABI 7900HT real-time PCR machine.

The term “elevated level” as used herein with respect to the level of TOP2A expression is any level that is greater than a reference level for TOP2A expression. The term “reference level” as used herein with respect to TOP2A expression is the level of TOP2A expression typically expressed by mammals free of aggressive prostate cancer. For example, a reference level of TOP2A expression can be the average level of TOP2A expression that is present in samples obtained from a random sampling of 50 males with prostate cancer who did not have systemic progression for at least seven years after having a prostatectomy. In some cases, a reference level can be the average level of TOP2A expression that is present in samples obtained from a random sampling of 50 males with a prostate cancer of Gleason score 7 who did not have systemic progression for at least seven years after having a prostatectomy.

It will be appreciated that levels from comparable samples are used when determining whether or not a particular level is an elevated level. For example, the average level of TOP2A expression present in bulk prostate tissue from a random sampling of mammals may be X units/g of prostate tissue, while the average level of TOP2A expression present in isolated prostate epithelial cells may be Y units/number of prostate cells. In this case, the reference level for TOP2A expression in bulk prostate tissue would be X units/g of prostate tissue, and the reference level for TOP2A expression in isolated prostate epithelial cells would be Y units/number of prostate cells. Thus, when determining whether or not the level of TOP2A expression in bulk prostate tissue is elevated, the measured level would be compared to the reference level for TOP2A expression in bulk prostate tissue. In some cases, the reference level of TOP2A expression can be a ratio of an expression value of TOP2A in a sample to an expression value of a control nucleic acid or polypeptide in the sample. A control nucleic acid or polypeptide can be any polypeptide or nucleic acid that has a minimal variation in expression level across various samples of the type for which the nucleic acid or polypeptide serves as a control. For example, GAPDH, HPRT, NDUFA7, and RPS16 nucleic acids or polypeptides can be used as control nucleic acids or polypeptides, respectively, in prostate samples.

An elevated level of TOP2A expression can be any level provided that the level is greater than a corresponding reference level for TOP2A expression. For example, an elevated level of TOP2A expression can be 1.5, 1.6, 1.7, 1.8, 1.9, 2.0, 2.2, 2.4, 2.6, 2.8, 3, 3.2, 3.4, 3.6, 3.8, 4, 4.2, 4.4, 4.6, 4.8, 5, 6, 7, 8, 9, 10, 15, 20, or more times greater than the reference level for TOP2A expression.

Any appropriate method can be used to determine the level of TOP2A expression present within a sample. For example, quantitative PCR, in situ hybridization, or microarray technology can be used to determine the level of TOP2A expression in a sample. In some cases, the level of TOP2A expression can be determined using polypeptide detection methods such as immunochemistry techniques. For example, antibodies specific for TOP2A polypeptides can be used to determine the polypeptide level of TOP2A in a sample.

Any appropriate type of sample can be used to evaluate the level of TOP2A expression including, without limitation, prostate cancer tissue. In addition, any appropriate method can be used to obtain a sample. For example, a prostate tissue sample (e.g., prostate cancer tissue sample) can be obtained by a tissue biopsy or following surgical resection. Once obtained, a sample can be processed prior to measuring the level of TOP2A expression. For example, a prostate tissue sample can be processed to extract RNA from the sample. Once obtained, the RNA can be evaluated to determine the level of TOP2A mRNA present. In some embodiments, nucleic acids present within a sample can be amplified (e.g., linearly amplified) prior to determining the level of TOP2A expression (e.g., using array technology). In another example, a prostate tissue sample can be frozen, and sections of the frozen tissue sample can be prepared on glass slides. The frozen tissue sections can be stored (e.g., at −80° C.) prior to analysis, or they can be analyzed immediately (e.g., by immunohistochemistry with an antibody specific for TOP2A polypeptides). In some cases, the level of a TOP2A nucleic acid or polypeptide can be evaluated in a prostate tissue sample obtained following a prostatectomy procedure.

Once the level of TOP2A expression in a sample from a mammal is determined, then the level can be compared to a reference level for TOP2A expression and used to evaluate the susceptibility of the mammal to a poor prostate cancer outcome. For a patient lacking ERG expression, a level of TOP2A expression in a sample from that patient that is higher than the corresponding reference level for TOP2A expression can indicate that that patient is susceptible to a poor outcome.

In some cases, the ERG expression status and level for TOP2A expression can be used in combination with one or more other factors to determine whether or not a mammal having prostate cancer is susceptible to a poor prostate cancer outcome. For example, the ERG expression status and level for TOP2A expression can be used in combination with the clinical stage, the serum PSA level, and/or the Gleason score of the prostate cancer to determine whether or not the mammal is likely to have to a poor outcome. Additional information about the mammal, such as information concerning genetic predisposition to develop cancer, SNPs, chromosomal abnormalities, gene amplifications or deletions, and/or post translational modifications, can also be used in combination with the ERG expression status and level for TOP2A expression to assess the aggressiveness and outcome of prostate cancer. In some cases, the ERG expression status and level for TOP2A expression can be used in combination with the Gleason score, preoperative PSA, seminal vesicle invasion, and margin status to determine whether or not the mammal is susceptible to a poor outcome (see, e.g., Blute et al., J Urol., 165(1):119-25 (2001)).

This document also provides methods and materials to assist medical or research professionals in identifying a mammal as being susceptible to a poor prostate cancer outcome. Medical professionals can be, for example, doctors, nurses, medical laboratory technologists, and pharmacists. Research professionals can be, for example, principle investigators, research technicians, postdoctoral trainees, and graduate students. A professional can be assisted by (1) determining a mammal's ERG expression status, (2) determining the level of TOP2A expression in a sample, and (3) communicating information about that status and level to that professional.

Any method can be used to communicate information to another person (e.g., a professional). For example, information can be given directly or indirectly to a professional. In addition, any type of communication can be used to communicate the information. For example, mail, e-mail, telephone, and face-to-face interactions can be used. The information also can be communicated to a professional by making that information electronically available to the professional. For example, the information can be communicated to a professional by placing the information on a computer database such that the professional can access the information. In addition, the information can be communicated to a hospital, clinic, or research facility serving as an agent for the professional.

The invention will be further described in the following examples, which do not limit the scope of the invention described in the claims.

### EXAMPLES

### Example 1

### TOP2A Polypeptide Levels are Predictive of Outcome in Surgically Treated ERG—Prostate Cancer Patients

### Methods and Materials

Using the Mayo Clinic Radical Prostatectomy database between the years 1994 to 2004, 140 men who developed systemic progression or died of prostate cancer within six years following RP were identified. These cases were matched with 117 men that did not develop systemic progression or die of prostate cancer with at least eight years of follow-up. Cases and controls were matched by a computerized score that included GS, pTNM stage, margin status, and preoperative serum PSA, age and year of surgery; and had similar frequencies in adjuvant treatment (Table 1).

TABLE 1
The clinical and pathologic features of
the patients in the case-control study.

| | Case | Control | p |
|---|---|---|---|
| Feature | (N = 140) | (N = 117) | value |
| Age at Surgery | | | 0.7 |
| Mean (SD) | 63.8 (7) | 64.1 (6.4) | |
| Median | 65 | 65 | |
| Range | (47-77) | (50-76) | |
| Preop PSA (ng/mL) | | | 0.996 |
| Median | 11.4 | 11.3 | |
| Q1, Q3 | 1.3 23.3 | 1.6 21.1 | |
| Range | (1.3 143) | (1.6-119) | |
| Gleason Score | | | 0.25 |
| 7 | 86 (61%) | 63 (54%) | |
| 8+ | 54 (39%) | 54 (46%) | |
| Pathologic Stage, 1997 TNM | | | 0.25 |
| T2aN0 | 8 (6%) | 11 (9%) | |
| T2bN0 | 21 (15%) | 21 (18%) | |
| T3aN0 | 31 (22%) | 30 (26%) | |
| T3b4N0 | 50 (36%) | 27 (23%) | |
| TxN+ | 30 (21%) | 28 (24%) | |
| Margin Positive | 88 (63%) | 78 (67%) | 0.53 |
| Ploidy | | | 0.01 |
| Diploid | 57 (41%) | 60 (51%) | |
| Tetraploid | 53 (38%) | 48 (41%) | |
| Aneuploid | 29 (21%) | 9 (8%) | |
| Adjuvant Hormonal Treatment | 55 (39%) | 46 (39%) | 0.99 |
| Adjuvant Radiation Treatment | 16 (11%) | 15 (13%) | 0.77 |

TOP2A polypeptide level was evaluated using standard immunohistochemical techniques. H&E tumor sections were reviewed from each patient, and the block with the highest Gleason score and greatest tumor content was selected for immunohistochemical staining using a monoclonal antibody to TOP2A, clone 3F6, at a dilution of 1:100 (Novacastra, Benton Lane, U.K.). The immunohistochemical stain was detected using the Dako Advance polymer-based detection system (Dako, Carpenteria, Calif., U.S.). The antigen localizes predominantly to the cell nucleus, and the immunohistochemical stain produced distinct and dark nuclear staining The immunohistochemical stains were reviewed, and the most intensely staining foci from Gleason patterns 4 and 5 were circled, ranging from 2.4 to 110 mm².

As described elsewhere (Parker et al., Cancer, 107:37-45 (2006)), each slide was scanned using a Slide Scanner (Bacus Laboratories, Inc., Lombard, Ill., U.S.). The system captures digital images at 480-752 pixel resolution at 40× magnification. Computer assisted immunohistochemical stain quantification was performed using IHC Score Software (Bacus Laboratories, Inc.) to obtain measurements of TOP2A-positive nuclear area and total nuclear area within the circled foci. TOP2A-positive nuclear area was expressed as a percentage of total tumor nuclear area and was assigned a score designated as Labeling Index (LI). All TOP2A stained sections required at least one intensely staining nucleus to be included in the study (internal positive control). After computer assisted quantification, TOP2A immunostained slides were reviewed and correlated with TOP2A LI to ensure appropriate quantification.

ERG mRNA levels were assessed as described elsewhere (Kosari et al., Clin. Cancer Res., 14:1734-1743 (2008)). Briefly, quantitative PCR was performed for ERG and TOP2A mRNA expression on the identical block used for immunohistochemistry to assess TOP2A polypeptide expression. H&E sections were reviewed, and the area of tumor circled. Ten-micron sections were taken from each block and prepared under RNase free conditions, and deparaffinized in xylene. Tumor was scraped from the slide based on the circled template from the H&E slide, and placed in 1.5-mL tubes containing digestion buffer (RecoverAll kit, Ambion, Austin, Tex.). Total RNA was isolated according to the RecoverAll RNA isolation procedure and treated with DNase using Ambion Turbo DNA free kit according to the manufacturer's instructions (Ambion). The amount of RNA was measured by the Quant-iT RiboGreen kit (Invitrogen, Carlsbad, Calif.). Reverse transcription (RT) was done using Superscript III First Strand Synthesis System (Invitrogen) and 500 ng of RNA in a 40-μL reaction volume.

Quantitative PCR was done on each sample by adding 12.5 ng total RNA equivalent cDNA to a 20-4 reaction volume for each gene using SYBR Green PCR Master Mix (Applied Biosystems, ABI, Foster City, Calif.) on the ABI 7900HT real-time PCR machine using the manufacturer's default cycling conditions. Primers for quantitative PCR were designed by using Primer Express software (ABI) to amplify a 70 to 85 base pair fragment of the Affymetrix (Santa Clara, Calif.) target sequence from microarray experiments described elsewhere (Kosari et al., Clin. Cancer Res., 14:1734-1743 (2008)).

Ct thresholds were set manually to allow reproducible Cts. Delta Cts were obtained by subtracting the Ct of the normalizing gene from the Ct of the test gene of the same RT reaction. The normalizing gene, 40S ribosomal protein S28 (RPS28), was chosen by assessment of the Affymetrix data from previous experiments for a gene exhibiting the most stable expression across all prostate samples, normal and cancer.

Case and control distributions were compared using chi-square test. ROC curve areas were estimated for TOP2A for all patients and stratified by ERG status. Standard errors for AUCs were computed by the Rank Correlation for Censored Data test (Herrell and Newson, Stata Journal, 6:309-334 (2006)). AUCs were also computed by R, using the somers2 functions tests.

### RESULTS

TOP2A Polypeptide Levels and Association with Outcome and ERG Status

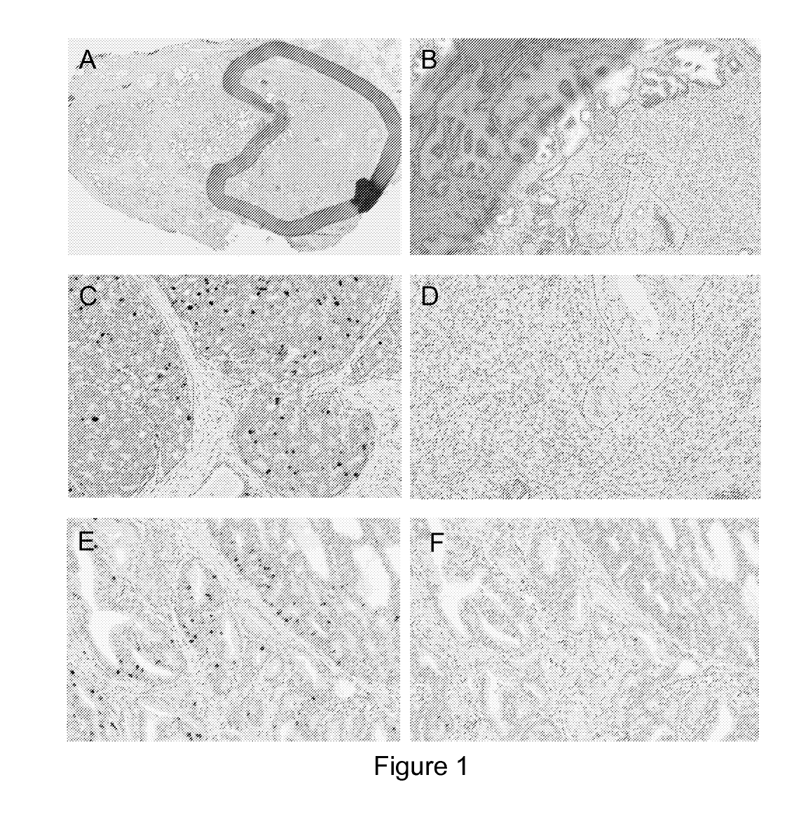

TOP2A immunohistochemical analysis revealed distinct and intense staining localized to the tumor cell nuclei. The evaluation of staining was restricted to the two highest Gleason patterns, 4 and 5. LIs mean±SD for cases and controls were 3.45±5.18 (range, 0.02-30.74) and 0.92±1.34 (range, 0.01-7.09), respectively (FIG. 1). ERG overexpression was present in 41% of cases and 48% of controls.

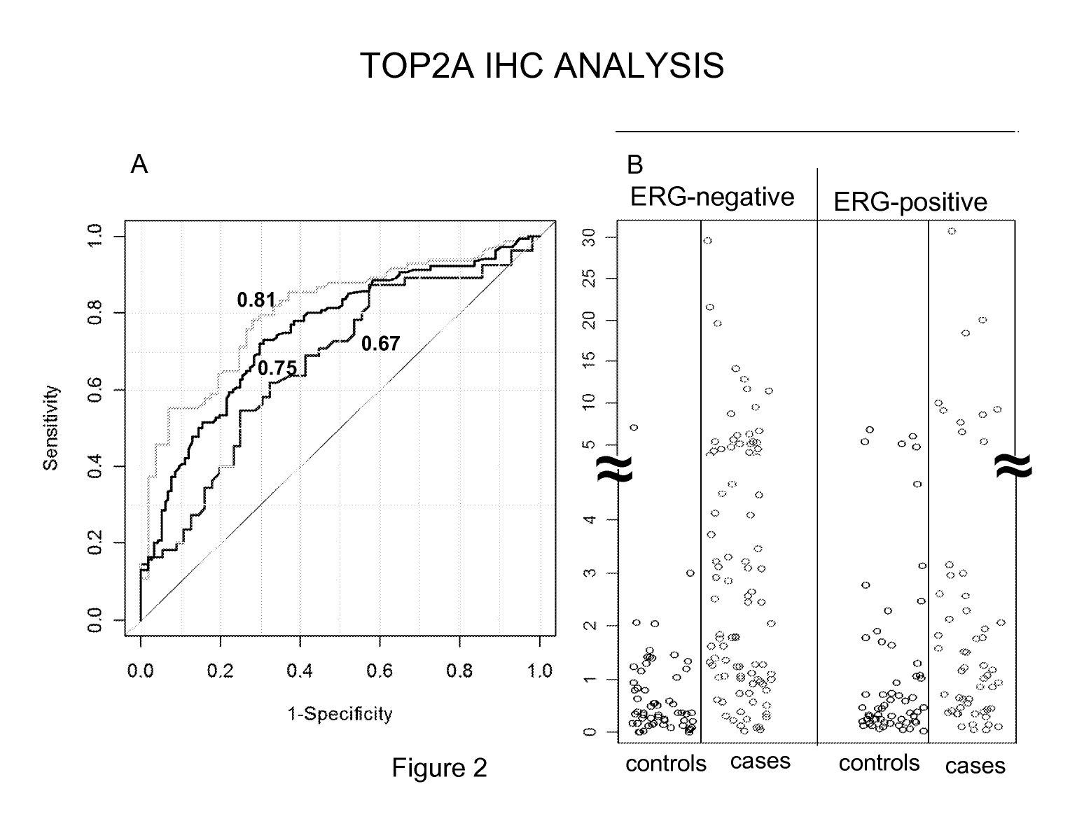

For all patients in this study, TOP2A polypeptide levels achieved an AUC of 0.75 (95% CI, 0.69 to 0.81). As the association of TOP2A LIs and systemic progression varied for ERG status (p=0.005 for interaction), the analysis was stratified by ERG status. The prognostic ability of TOP2A for systemic progression and prostate cancer death was significantly better in men with ERG(−) cancers than in men with ERG(+) cancers with AUC 0.81 (95% CI, 0.74 to 0.88) versus 0.67 (95% CI, 0.57 to 0.77) (FIG. 2).

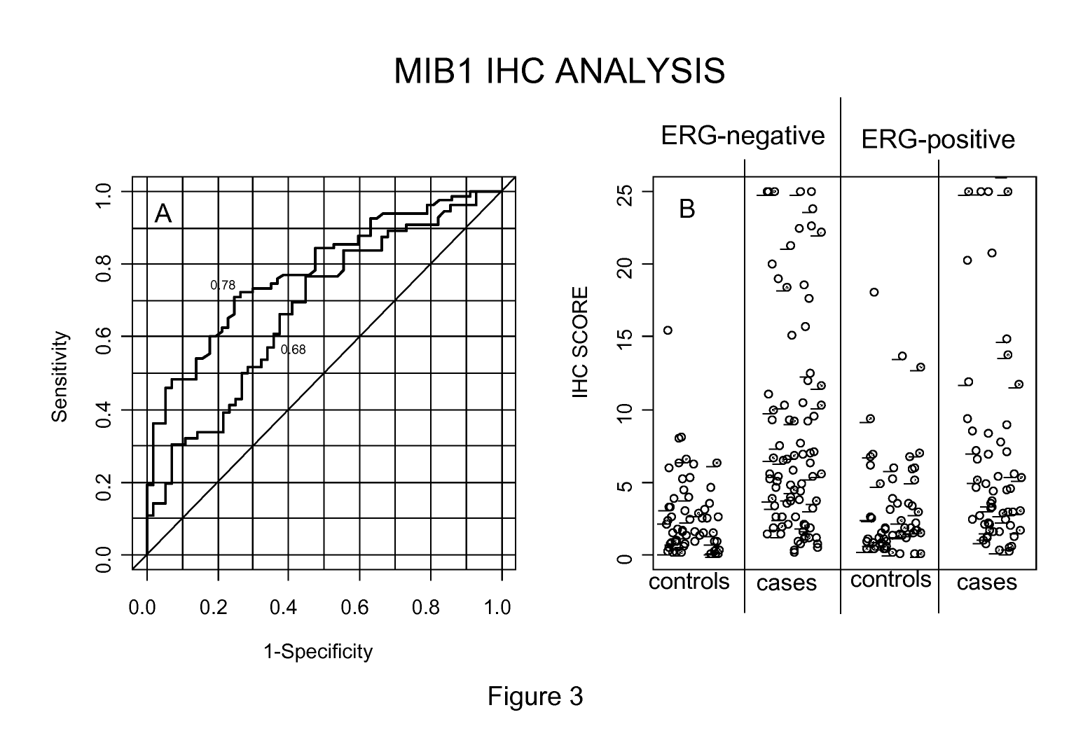

Similarly, when analysis was stratified by ERG status, the prognostic ability of MIB-1 LIs for SP was also significantly better in men with ERG(−) cancers than in men with ERG(+) cancers, with an AUC of 0.78 (95% CI, 0.71 to 0.85) vs. 0.68 (95% CI, 0.58 to 0.78) (FIG. 3).

TOP2A Polypeptide Levels, ERG Status, and Association with Time to Systemic Progression

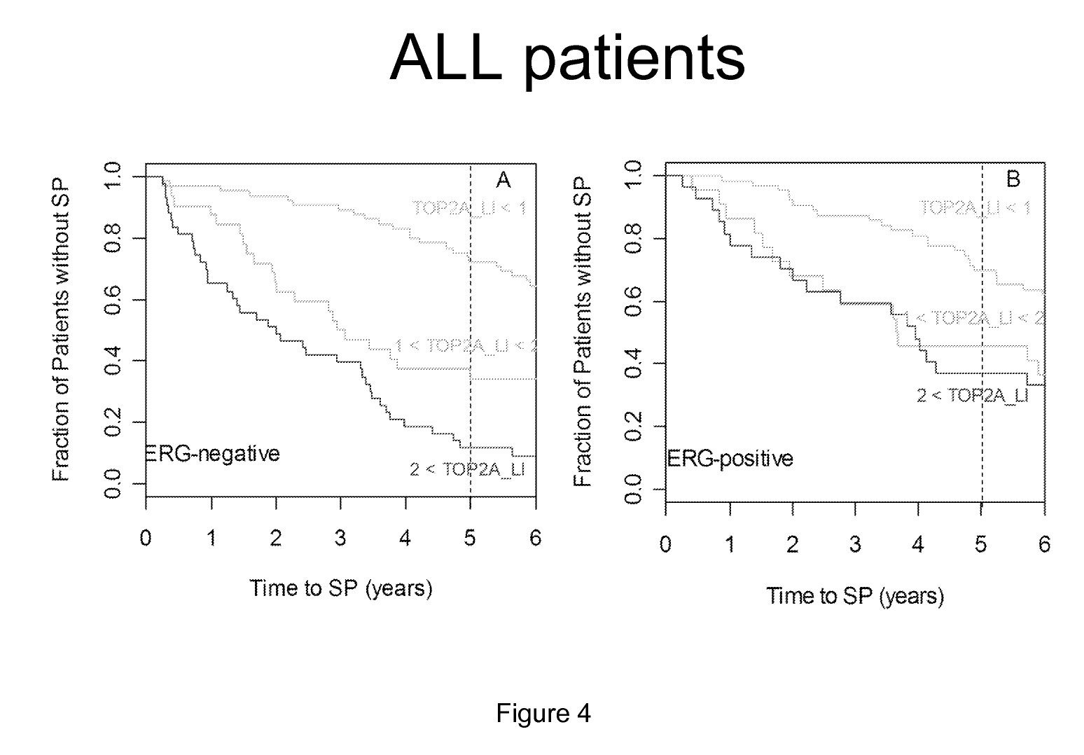

For all patients in this study, the relationship between time to systemic progression following RP, TOP2A LIs, and ERG status was examined. The time to systemic progression was related to both TOP2A polypeptide level and ERG status (FIG. 4). Two cutpoints were investigated for the TOP2A LIs of 1 and 2 that based on the scatter plot distribution separated cases and controls, and the percent of patients that developed systemic progression following RP were plotted with respect to ERG status. TOP2A LIs clearly separated these patients into groups that differ significantly in the time to systemic progression. The curves related to systemic progression were generated from a case control study design, and are not representative of the overall population of men with high risk prostate cancer. Nonetheless, the curves reveal a significant difference in the 5-year rate of systemic progression based on ERG status and TOP2A LI (FIG. 4).

The patients with ERG(−) cancers can be separated in three groups based on TOP2A LIs: slow progressors TOP2A LI<1, intermediate progressors 1<TOP2A LI<2, and rapid progressors, TOP2A LI>2 as illustrated in FIG. 4A. There was a statistically significant difference in the time to progression between the three groups (slow to rapid progressors, p<0.0001, slow to intermediate progressors, p<0.001, and intermediate to rapid progressors, p<0.015). When patients with ERG(+) cancer were separated in the three risk groups, the difference between intermediate and rapid rates of progression was not significant (p=0.7) as illustrated in FIG. 4B. The cutpoint of TOP2A LI of 1 separated ERG(+) patients into two groups with significantly different times to systemic progression (p<0.001).

### TOP2A Polypeptide Levels, ERG Status, and Survival Related to Adjuvant Androgen Deprivation Therapy

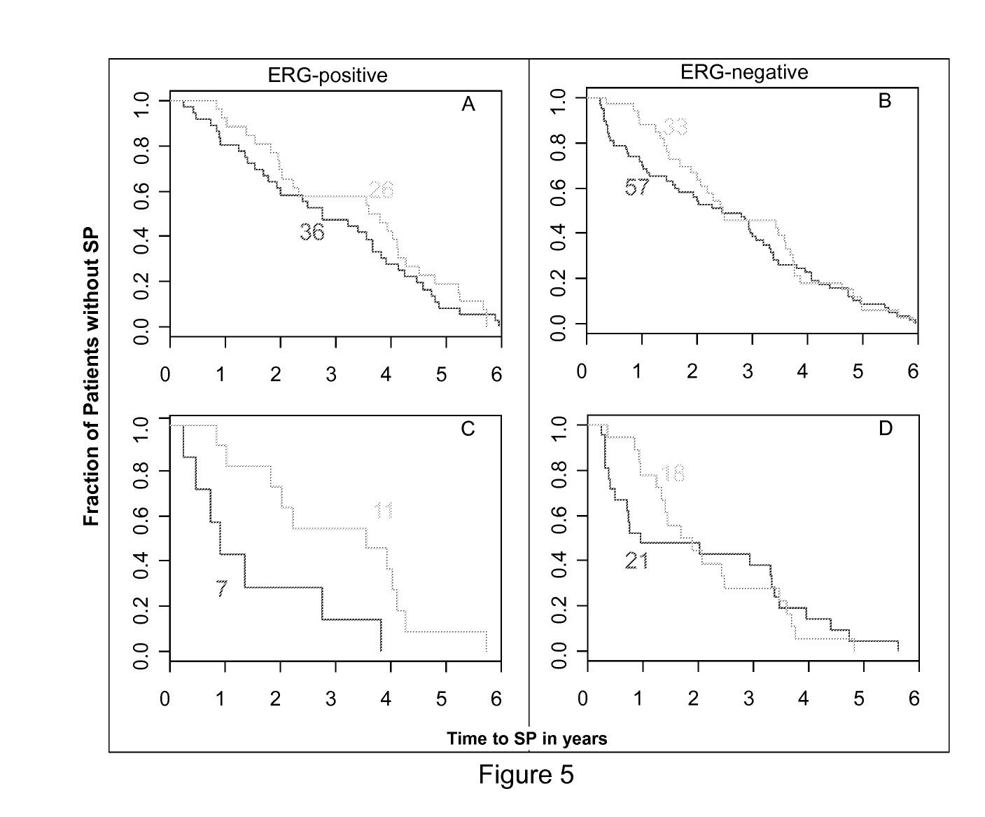

To determine the affects of adjuvant androgen deprivation, TOP2A polypeptide expression and ERG status were examined in patients that received and did not receive adjuvant therapy postoperatively and prior to systemic progression (cases only). Although there were differences in time to systemic progression based on ERG(+) and ERG(−) tumor status, these differences did not reach statistical significance (FIGS. 5A and 5B). However, for patients with TOP2A LI>2 and ERG(+) tumors, adjuvant androgen deprivation therapy resulted in a significantly slower time to systemic progression (p=0.015). This affect was not seen in ERG(−) tumors (FIGS. 5C and 5D).

The results provided herein demonstrate that the polypeptide expression of TOP2A identifies men at greatest risk of systemic progression and prostate cancer death following RP. In addition, in men that developed systemic progression, the results provided herein demonstrate that TOP2A polypeptide expression was able to stratify men based on their ERG status into groups that differed significantly in their time to systemic progression. Finally, although patient numbers are small, in men with ERG(+) tumors and a TOP2A LI>2, postoperative adjuvant hormonal deprivation therapy appeared to provide a benefit in delaying the time to systemic progression.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-19. (canceled)

20. A method for assessing prostate cancer, wherein said method comprises:
(a) obtaining a prostate cancer tissue sample from a mammal having prostate cancer,
(b) performing a quantitative polymerase chain reaction to detect reduced or no ERG expression in said tissue sample,
(c) staining a section of said tissue sample using an anti-TOP2A antibody to identify an intensely staining foci region,
(d) obtaining measurements of a TOP2A-positive nuclear area and a total nuclear area within said region,
(e) determining that said section has a labeling index greater than 2, wherein said labeling index is the percentage of said total tumor nuclear area that is said TOP2A-positive nuclear area, and
(f) classifying said mammal as being susceptible to a poor prostate cancer outcome based at least in part on said reduced or no ERG expression and the presence of said labeling index.

21. The method of claim 20, wherein said mammal is a human.

22. The method of claim 20, wherein said reduced or no ERG expression is detected based on a lack of ERG expression.

23. The method of claim 20, wherein said poor prostate cancer outcome comprises systemic progression within five years of prostatectomy.

24. The method of claim 20, wherein said poor prostate cancer outcome comprises prostate cancer death.

25. The method of claim 20, wherein said mammal comprises a human treated by radical prostatectomy.
